(19) United States
(12) Patent Application Publication
(10) Pub. No.: US 2008/0166411 A1
(43) Pub. Date: Jul. 10, 2008

(54) Injectable Depot Formulations And Methods For Providing Sustained Release Of Poorly Soluble Drugs Comprising Nanoparticles

(75) Inventors: Jaymin C. Shah, Waterford, CT (US); Parah Suresh Shah, Groton, CT (US); Dawn Renee Wagner, Groton, CT (US); Peter Wisniecki, Coventry, CT (US)

Correspondence Address: PFIZER INC;Steve T. Zelson, 150 EAST 42ND STREET, 5TH FLOOR - STOP 49, NEW YORK, NY, 10017-5612, US

(73) Assignee: Pfizer Inc

(21) Appl. No.: 11/911,432

(22) PCT Filed: Apr. 10, 2006
(86) PCT No.: PCT/IB06/01011

§ 371 Date: Oct. 12, 2007

(51) Int. Cl. A61K 9/16

(52) U.S. Cl. 424/489

(57) ABSTRACT

Pharmaceutical formulations comprising: a compound of low water solubility, having a maximum average particle size; a carrier; and at least two surface stabilizers are disclosed. The present invention also comprises methods of treating various conditions with such a formulation and processes for making such a formulation.

### FIELD OF THE INVENTION

The present invention comprises a pharmaceutical formulation comprising: a compound of low water solubility, having a maximum average particle size; a carrier; and at least two surface stabilizers. The present invention also comprises methods of treatment with such a formulation and processes for making such a formulation.

### BACKGROUND OF THE INVENTION

Unfortunately, many useful drugs have low solubility in water and, therefore, are difficult to formulate at convenient concentrations as solutions in an aqueous vehicle. Even when a suitable solvent is found as a vehicle for such a drug, there is often a tendency, particularly for a crystalline drug of low water solubility, to precipitate out of solution and/or crystallize when the drug comes in contact with water, for example in the aqueous environment of the gastrointestinal tract. Such precipitation and/or re-crystallization can offset or reduce the potential rapid onset benefits sought by formulating the drug as a solution.

One problem with drugs and drug candidates of low water solubility is that it is difficult to evaluate their bioefficacy. Producing formulations with high bioavailability is even more difficult with poorly water soluble drugs. Special non-aqueous formulations can be used, but they are not really patient friendly.

There have been many formulations developed specifically for increasing the aqueous solubility of poorly soluble drugs. Solid extrudates (dispersion/solution) are an attractive approach, but have not been used widely due to the required increases in temperature beyond melting points of drugs and polymers. Only a limited number of commercial formulations are available.

Oral delivery of peptide and protein drugs has been one of the most active research areas in drug delivery. Insulin has been used as a model drug in most studies dealing with oral administration of protein drugs. A number of approaches have been developed and, in fact, many of them showed transient control of glucose levels. While it may be possible to lower the glucose level for a short time by oral administration of insulin, the issues of reproducibility and the kinetics of absorption have to be very carefully examined. No report in the literature has shown the repeated administration of insulin resulting in effective control of the glucose level, and has induced the insulin effect at the right time. The bioavailability is also a great concern in the study of oral protein delivery. To date, all studies have indicated that only a very small fraction (<10%) of the total dose of orally administered insulin is bioavailable. A major concern is the waste of more than 90% of the administered insulin.

Targeted delivery of drugs to the colon has become popular in recent years because the delivery of drugs labile to acid and enzymes in a region that is less hostile metabolically results in enhanced absorption of certain drugs. Drugs with poor solubility may not dissolve in the colon where there is not as much fluid as in the upper portion of the GI tract. The current technologies are mainly focused on delayed release, that is, no release until the dosage form reaches the colon. The time-delay approach relies on drug release following a predetermined lag time and would therefore be less dependable.

Nanotechnology presents an opportunity to increase the bioavailability of drug particles. A decrease in particle size results in increased surface area, which may result in faster dissolution, normally by a small order of magnitude. In some cases, this may be enough to result in increased bioavailability. However, faster dissolution may not be sufficient to overcome exposure to acid and enzymes in the gut. Additionally, as in the case with oral insulin, this exposure may require higher dosing of the drug, resulting in unnecessary and potentially undesirable subject exposure to breakdown products as well as create significant waste.

A depot formulation is specially formulated to provide slow absorption of the drug from the site of administration, often keeping therapeutic levels of the drug in the patient's system for days or weeks at a time. Alternatively, a depot formulation may provide convenience for a patient in need of chronic medication. By delivering drug without exposure to the GI tract, the potential issue of drug degradation is avoided. Moreover, a depot formulation may provide better compliance due to the infrequent dosing regimen and convenience. Additional characteristics of a depot formulation that will enhance patient compliance are good local tolerance at the injection site and ease of administration. Good local tolerance means minimal irritation and inflammation at the site of injection; ease of administration refers to the size of needle and length of time required to administer a dose of a particular drug formulation.

U.S. Pat. No. 6,232,304 (granted May 15, 2001) describes a ziprasidone salt solubilized with cyclodextrins for an immediate release intramuscular injection formulation.

U.S. Pat. No. 6,150,366 (granted Nov. 21, 2000) describes a pharmaceutical composition describing crystalline ziprasidone and a carrier.

U.S. Pat. No. 6,267,989 (granted Jul. 31, 2001) describes a water-insoluble crystalline drug to which a surface modifier is adsorbed in an amount sufficient to maintain a defined particle size.

U.S. Pat. No. 5,145,684 (granted Sep. 8, 1992) describes low solubility crystalline drug substances to which a surface modifier is adsorbed in an amount sufficient to maintain a defined particle size.

U.S. Pat. No. 5,510,118 (granted Apr. 23, 1996) describes a homogenization process to obtain sub-micron drug substances without milling media.

U.S. Pat. No. 5,707,634 (granted Jan. 13, 1998) describes a method precipitating a crystalline solid from liquid.

U.S. Pat. No. 5,314,685 (granted May 24, 1994) describes techniques for solubilizing hydrophobic compounds with low water solubility.

U.S. Pat. No. 4,992,271 (granted Feb. 12, 1991) describes techniques for solubilizing hydrophobic compounds with low water solubility.

U.S. Patent Application No. 60/585,411 (filed Jul. 1, 2004) describes a high pressure homogenization method to prepare nanoparticles.

WO 00/18374 (filed Oct. 1, 1999) describes a controlled release nanoparticle composition.

WO 00/09096 (filed Aug. 12, 1999) describes an injectable nanoparticle formulation of naproxen.

Certain potential pharmaceuticals are hydrophobic and typically have low aqueous solubility and hence low oral bioavailability. It is believed that the invention provides an acceptable depot formulation of low water solubility drug nanoparticles, which is efficacious and has an acceptable injection volume. In addition to enhancing patient compliance, a nanoparticle depot formulation of a low solubility drug may reduce overall exposure to the drug compared to oral capsules while providing sufficient exposure to ensure efficacy.

### SUMMARY OF THE INVENTION

In one embodiment, the present invention comprises a pharmaceutical formulation suitable for use as a depot formulation for administration via intramuscular or subcutaneous injection. The formulation comprises (1) a low solubility drug or pharmaceutically acceptable salt thereof; (2) a pharmaceutically acceptable carrier; and (3) at least two surface stabilizers. The formulations of the invention may, for example, comprise from two to ten surface stabilizers, preferably two to five surface stabilizers. In another embodiment, the formulation consists of two surface stabilizers. In another embodiment, the formulation consists of three surface stabilizers. In still another embodiment, the formulation consists of two surface stabilizers and a bulking agent.

In another embodiment, the present invention comprises processes for preparing such a formulation.

In another embodiment, the present invention comprises the use of such a composition as a medicament in the treatment of a wide range of disorders. In yet another embodiment, the present invention comprises methods of treating these disorders.

### DETAILED DESCRIPTION OF THE INVENTION

This detailed description of embodiments is intended only to acquaint others skilled in the art with Applicants' inventions, its principles, and its practical application so that others skilled in the art may adapt and apply the inventions in their numerous forms, as they may be best suited to the requirements of a particular use. These inventions, therefore, are not limited to the embodiments described in this specification, and may be variously modified.

### A. ABBREVIATIONS AND DEFINITIONS

TABLE A-1
Abbreviations

| | |
|---|---|
| API | Active pharmaceutical ingredient |
| AUC | Area under the curve |
| Cₘₐₓ | Maximum serum concentration of compound |
| CPB | Cloud point booster |
| DLS | Dynamic light scattering |
| EPS | Extrapyramidal symptoms |
| D[4,3] | Volume average diameter |
| F | bioavailability |
| FB | Free base |
| Form. | formulation |
| Gy | Gray - a measure of irradiation dose |
| H | hours |
| HCl | Hydrochloride salt |
| IM | intramuscular |
| IR | Immediate release |
| Mes | Mesylate salt |
| Ml | Milliliter |
| MW | Molecular weight |
| ng | nanograms |
| nm | Nanometer |
| NMP | N-methyl-pyrrolidone |
| PEG | Polyethylene glycol |
| PK | Pharmacokinetics |
| PVA | polyvinylalcohol |
| PVP | polyvinylpyrrolidone |
| PVP C15 | A particular grade of PVP |
| PVP K30 | A particular grade of PVP |
| RPM | Revolutions per minute |
| RPS | Reduced particle size |
| SA/V | Surface area to volume ratio |
| SBECD | sulfobutylether-β-cyclodextrin |
| SLS | Sodium lauryl sulfate |
| t_{1/2} | Terminal elimination phase half-life |
| Tₘₐₓ | Time to maximum serum concentration of compound |
| v/v | Volume by volume |
| VDₛₛ | Volume of distribution at steady state |
| w/v | Weight by volume |
| Z-Com. | Ziprasidone compound |

The term “compound” refers to a form of a therapeutic or diagnostic agent which is a component of an injectable depot formulation. The compound may be a pharmaceutical, including, without limitation, biologics such as proteins, peptides and nucleic acids or a diagnostic, including, without limitation, contrast agents. In one embodiment, the compound is crystalline. In another embodiment, the compound is amorphous. In another embodiment, the compound is a mixture of crystalline and amorphous forms. In another embodiment, the compound has low water solubility. In another embodiment, the logP of the compound is at least about 3 or greater. In another embodiment, the compound has a high melting point. A high melting compound is one with a melting point greater than about 130 degrees Celsius.

A compound having “low water solubility” refers to any compound having a solubility in water, measured at 37° C., not greater than about 10 mg/ml. In another embodiment, the measured solubility is not greater than about 1 mg/ml. In another embodiment, the measured solubility is not greater than about 0.1 mg/ml. A synonymous term is “low aqueous solubility.” Solubility in water for many drugs can be readily determined from standard pharmaceutical reference books, for example The Merck Index, 13th ed., 2001 (published by Merck & Co., Inc., Rahway, N.J.); the United States Pharmacopoeia, 24th ed. (USP 24), 2000; The Extra Pharmacopoeia, 29th ed., 1989 (published by Pharmaceutical Press, London); and the Physicians Desk Reference (PDR), 2005 ed. (published by Medical Economics Co., Montvale, N.J.).

For example, individual compounds of low solubility as defined herein include those drugs categorized as “slightly soluble”, “very slightly soluble”, “practically insoluble” and “insoluble” in USP 24, pp. 2254-2298; and those drugs categorized as requiring 100 ml or more of water to dissolve 1 g of the drug, as listed in USP 24, pp. 2299-2304.

Exemplary compounds, include, without limitation; compounds from the following classes: abortifacients, ACE inhibitors, α- and β-adrenergic agonists, α- and β-adrenergic blockers, adrenocortical suppressants, adrenocorticotropic hormones, alcohol deterrents, aldose reductase inhibitors, aldosterone antagonists, anabolics, analgesics (including narcotic and non-narcotic analgesics), androgens, angiotensin II receptor antagonists, anorexics, antacids, anthelminthics, antiacne agents, antiallergics, antialopecia agents, antiamebics, antiandrogens, antianginal agents, antiarrhythmics, antiarteriosclerotics, antiarthritic/antirheumatic agents, antiasthmatics, antibacterials, antibacterial adjuncts, anticholinergics, anticoagulants, anticonvulsants, antidepressants, antidiabetics, antidiarrheal agents, antidiuretics, antidotes to poison, antidyskinetics, antieczematics, antiemetics, antiestrogens, antifibrotics, antiflatulents, antifungals, antiglaucoma agents, antigonadotropins, antigout agents, antihistaminics, antihyperactives, antihyperlipoproteinemics, antihyperphosphatemics, antihypertensives, antihyperthyroid agents, antihypotensives, antihypothyroid agents, anti-inflammatories, antimalarials, antimanics, antimethemoglobinemics, antimigraine agents, antimuscarinics, antimycobacterials, antineoplastic agents and adjuncts, antineutropenics, antiosteoporotics, antipagetics, antiparkinsonian agents, antipheochromocytoma agents, antipneumocystis agents, antiprostatic hypertrophy agents, antiprotozoals, antipruritics, antipsoriatics, antipsychotics, antipyretics, antirickettsials, antiseborrheics, antiseptics/disinfectants, antispasmodics, antisyphylitics, antithrombocythemics, antithrombotics, antitussives, antiulceratives, antiurolithics, antivenins, antiviral agents, anxiolytics, aromatase inhibitors, astringents, benzodiazepine antagonists, bone resorption inhibitors, bradycardic agents, bradykinin antagonists, bronchodilators, calcium channel blockers, calcium regulators, carbonic anhydrase inhibitors, cardiotonics, CCK antagonists, chelating agents, cholelitholytic agents, choleretics, cholinergics, cholinesterase inhibitors, cholinesterase reactivators, CNS stimulants, contraceptives, debriding agents, decongestants, depigmentors, dermatitis herpetiformis suppressants, digestive aids, diuretics, dopamine receptor agonists, dopamine receptor antagonists, ectoparasiticides, emetics, enkephalinase inhibitors, enzymes, enzyme cofactors, estrogens, expectorants, fibrinogen receptor antagonists, fluoride supplements, gastric and pancreatic secretion stimulants, gastric cytoprotectants, gastric proton pump inhibitors, gastric secretion inhibitors, gastroprokinetics, glucocorticoids, α-glucosidase inhibitors, gonad-stimulating principles, growth hormone inhibitors, growth hormone releasing factors, growth stimulants, hematinics, hematopoietics, hemolytics, hemostatics, heparin antagonists, hepatic enzyme inducers, hepatoprotectants, histamine H₂ receptor antagonists, HIV protease inhibitors, HMG CoA reductase inhibitors, immunomodulators, immunosuppressants, insulin sensitizers, ion exchange resins, keratolytics, lactation stimulating hormones, laxatives/cathartics, leukotriene antagonists, LH-RH agonists, lipotropics, 5-lipoxygenase inhibitors, lupus erythematosus suppressants, matrix metalloproteinase inhibitors, mineralocorticoids, miotics, monoamine oxidase inhibitors, mucolytics, muscle relaxants, mydriatics, narcotic antagonists, neuroprotectives, nootropics, ovarian hormones, oxytocics, pepsin inhibitors, pigmentation agents, plasma volume expanders, potassium channel activators/openers, progestogens, prolactin inhibitors, prostaglandins, protease inhibitors, radio-pharmaceuticals, 5α-reductase inhibitors, respiratory stimulants, reverse transcriptase inhibitors, sedatives/hypnotics, serenics, serotonin noradrenaline reuptake inhibitors, serotonin receptor agonists, serotonin receptor antagonists, serotonin uptake inhibitors, somatostatin analogs, thrombolytics, thromboxane A₂ receptor antagonists, thyroid hormones, thyrotropic hormones, tocolytics, topoisomerase I and II inhibitors, uricosurics, vasomodulators including vasodilators and vasoconstrictors, vasoprotectants, xanthine oxidase inhibitors, and combinations thereof.

Examples of suitable compounds include, without limitation, acetohexamide, acetylsalicylic acid, alclofenac, allopurinol, atropine, benzthiazide, carprofen, celecoxib, chlordiazepoxide, chlorpromazine, clonidine, codeine, codeine phosphate, codeine sulfate, deracoxib, diacerein, diclofenac, diltiazem, estradiol, etodolac, etoposide, etoricoxib, fenbufen, fenclofenac, fenprofen, fentiazac, flurbiprofen, griseofulvin, haloperidol, ibuprofen, indomethacin, indoprofen, ketoprofen, lorazepam, medroxyprogesterone acetate, megestrol, methoxsalen, methylprednisone, morphine, morphine sulfate, naproxen, nicergoline, nifedipine, niflumic, oxaprozin, oxazepam, oxyphenbutazone, paclitaxel, palperidone, phenindione, phenobarbital, piroxicam, pirprofen, prednisolone, prednisone, procaine, progesterone, pyrimethamine, risperidone, rofecoxib, sulfadiazine, sulfamerazine, sulfisoxazole, sulindac, suprofen, temazepam, tiaprofenic acid, tilomisole, tolmetic, valdecoxib and ziprasidone.

Further exemplary compounds include, without limitation, Acenocoumarol, Acetyldigitoxin, Anethole, Anileridine, Benzocaine, Benzonatate, Betamethasone, Betamethasone Acetate, Betamethasone Valerate, Bisacodyl, Bromodiphenhydramine, Butamben, Chlorambucil, Chloramphenicol, Chlordiazepoxide, Chlorobutanol, Chlorocresol, Chlorpromazine, Clindamycin Palmitate, Clioquinol, Cortisone Acetate, Cyclizine Hydrochloride, Cyproheptadine Hydrochloride, Demeclocycline, Diazepam, Dibucaine, Digitoxin, Dihydroergotamine Mesylate, Dimethisterone, Disulfuram, Docusate Calcium, Docusate Sodium, Dihydrogesterone, Enalaprilat, Ergotamine Tartrate, Erythromycin, Erythromycin Estolate, Flumethasone Pivalate, Fluocinolone Acetonide, Fluorometholone, Fluphenazine Enanthate, Flurandrenolide, Guaifenesin, Halazone, Hydrocortisone, Levothyroxine Sodium, Methyclothiazide, Miconazole, Miconazole Nitrate, Nitrofurazone, Nitromersol, Oxazepam, Pentazocine, Pentobarbital, Primidone, Quinine Sulfate, Stanozolol, Sulconazole Nitrate, Sulfadimethoxine, Sulfaethidole, Sulfamethizole, Sulfamethoxazole, Sulfapyridine, Testosterone, Triazolam, Trichlormethiazide, and Trioxsalen.

The term “surface stabilizer” as used herein, unless otherwise indicated, refers to a molecule that: (1) is adsorbed on the surface of a compound; (2) otherwise physically adheres to the surface of a compound; or (3) remains in solution with a compound, acting to maintain the effective particle size of the compound. A surface stabilizer does not chemically react (i.e. form a covalent bond) with the drug substance (compound). A surface stabilizer also does not necessarily form covalent crosslinkages with itself or other surface stabilizers in a formulation and/or when adsorbed onto compound surfaces. In a preferred embodiment of the invention, a surface stabilizer on the surface of a compound or otherwise in a formulation of the invention is essentially free of covalent crosslinkages.

In one embodiment, a first surface stabilizer is present in an amount sufficient to maintain an effective average particle size of the compound. In a second embodiment, one or more surface stabilizers are present in an amount sufficient to maintain an effective particle size of the compound. In another embodiment, a surface stabilizer is a surfactant. In another embodiment, a surface stabilizer is a crystallization inhibitor.

The term “surfactant” refers to amphipathic molecules that consist of a non-polar hydrophobic portion, exemplified by a straight or branched hydrocarbon or fluorocarbon chain containing 8-18 carbon atoms, which is attached to a polar or ionic portion (hydrophilic). The hydrophilic portion may be nonionic, ionic or zwitterionic and accompanied by counter ions. There are several classes of surfactants: anionic, cationic, amphoteric, nonionic and polymeric. In the case of nonionic and polymeric surfactants, a single surfactant may be properly classified as a member of both categories. An exemplary group of surfactants that may be properly classified in this manner are the ethylene oxide-propylene oxide co-polymers, referred to as Pluronics® (Wyandotte), Synperonic PE® (ICI) and Poloxamers® (BASF). Polymers such as HPMC and PVP may be classified as polymeric surfactants. Exemplary classes of surfactants include, without limitation: carboxylates, sulphates, sulphonates, phosphates, sulphosuccinates, isethionates, taurates, quaternary ammonium compounds, N-alkyl betaines, N-alkyl amino propionates, alcohol ethoxylates, alkyl phenol ethoxylates, fatty acid ethoxylates, monoalkaolamide ethoxylates, sorbitan ester ethoxylates, fatty amine ethoxylates, ethylene oxide-propylene oxide co-polymers, glycerol esters, glycol esters, glucosides, sucrose esters, amino oxides, sulphinyl surfactants, polyoxyethylene allyl ethers, polyoxyethylene alkyl ethers, polyglycolized glycerides, short-chain glyceryl mono-alkylates, alkyl aryl polyether sulfonate, polyoxyethylene fatty acid esters, polyoxyethylene fatty acid ethers, polyoxyethylene stearates, copolymers of vinylacetate and vinylalcohol, and random copolymers of vinyl acetate and vinyl pyrrolidone.

Exemplary surfactants, include, without limitation: dodecyl hexaoxyethylene glycol monoether, sorbitan monolaurate, sorbitan monopalmitate, sorbitan monostearate, sorbitan mono-oleate, sorbitan tristearate, sorbitan trioleate, polyoxyethylene (20) sorbitan monolaurate, polyoxyethylene (20) sorbitan monopalmitate, polyoxyethylene (20) sorbitan monostearate, polyoxyethylene (20) sorbitan mono-oleate, polyoxyethylene (20) sorbitan tristearate, polyoxyethylene (20) sorbitan trioleate, linolin, castor oil ethoxylates, Pluronic® F108, Pluronic® F68, Pluronic® F127, benzalkonium chloride, colloidal silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethylcellulose, phthalate, noncrystalline cellulose, magnesium aluminate silicate, triethanolamine, polyvinyl alcohol (PVA), tyloxapol®, polyvinylpyrrolidone (PVP), sodium 1,4-bis(2-ethylhexyl) sulfosuccinate, sodium lauryl sulfate (SLS), polyoxyethylene (35) castor oil, polyethylene (60) hydrogenated castor oil, alpha tocopheryl polyethylene glycol 1000 succinate, glyceryl PEG 8 caprylate/caprate, PEG 32 glyceryl laurate, dodecyl trimethyl ammonium bromide, Aerosol OT®, Tetronic 908®, dimyristoyl phophatidyl glycerol, dioctylsulfosuccinate (DOSS), Tetronic 1508®, Duponol P®, Tritons X-200®, Crodestas F-110®, p-isononylphenoxypoly-(glycidol), SA90HCO, decanoyl-N-methylglucamide, n-decyl β-D-glucopyranoside, n-decyl β-D-maltopyranoside, n-dodecyl β-D-glucopyranoside, n-dodecyl β-D-maltoside, heptanoyl-N-methylglucamide, n-heptyl-β-D-glucopyranoside, n-heptyl β-D-thioglucoside, n-hexyl β-D-glucopyranoside, nonanoyl-N-methylglucamide, n-noyl β-D-glucopyranoside, octanoyl-N-methylglucamide, n-octyl-β-D-glucopyranoside, octyl β-D-thioglucopyranoside, dextrin, guar gum, starch, Plasdone® S630, Kollidone® VA 64, polyvinyl alcohol, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium®-15), distearyldimonium chloride (Quaternium®-5), dodecyl dimethyl ethylbenzyl ammonium chloride(Quaternium®-14), Quaternium®-22, Quaternium®-26, Quaternium®-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, 7 myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

The term “ethylene oxide-propylene oxide copolymers” refers to four types of nonionic block copolymers, of which Pluronic® F108 is one, as described in Table A-2, immediately below:

| Formula | Components of block copolymer |
|---|---|
| (EO)ₙ(PO)ₘ(EO)ₙ | Ethylene oxide-propylene oxide copolymer prepared by reaction of |
| | poly(oxypropylene glycol) (difunctional) with ethylene oxide |
| | Ethylene oxide-propylene oxide copolymer prepared by reaction of |
| | poly(oxypropylene glycol) (difunctional) with mixed ethylene oxide and |
| | propylene oxide, giving block copolymers |
| (PO)ₙ(EO)ₘ(PO)ₙ | Ethylene oxide-propylene oxide copolymer prepared by reaction of |
| | poly(ethylene glycol) (difunctional) with propylene oxide |
| | Ethylene oxide-propylene oxide copolymer prepared by reaction of |
| | poly(ethylene glycol) (difunctional) with mixed ethylene oxide and |
| | propylene oxide, giving block copolymers |

wherein m and n are varied systematically in each formula

The term “Pluronic® F108” refers to the polyoxyethylene-polyoxypropylene block copolymer that conforms generally to the formula HO[CH₂CH₂O]ₙ[CH(CH₃)CH₂O]ₘ[CH₂CH₂O]ₙH in which the average values of n, m and n are respectively 128, 54 and 128.

The use of trade names herein is not intended to limit suitable species for the invention to those produced or sold by any one particular manufacturer, but instead to assist in defining embodiments of the invention.

The term “crystallization inhibitor” refers to a polymer or other substances that can substantially inhibit precipitation and/or crystallization of a poorly water-soluble drug. In one embodiment, a polymeric surfactant is a crystallization inhibitor. In another embodiment, the crystallization inhibitor is a cellulosic or non-cellulosic polymer and is substantially water-soluble. In another embodiment, the crystallization inhibitor is HPMC. In another embodiment, a crystallization inhibitor is polyvinylpyrrolidone (PVP).

It will be understood that certain polymers are more effective at inhibiting precipitation and/or crystallization of a selected poorly water soluble drug than others, and that not all polymers inhibit precipitation and/or crystallization as described herein of every poorly water-soluble drug. Whether a particular polymer is useful as a crystallization inhibitor for a particular poorly water soluble drug according to the present invention can be readily determined by one of ordinary skill in the art, for example according to Test I, depicted in Table A-3:

TABLE A-3
Method to Test Crystallization Inhibitors for Efficacy

| | |
|---|---|
| Step 1 | A suitable amount of the drug is dissolved in a solvent (e.g., ethanol, dimethyl |
| | sulfoxide or, where the drug is an acid or base, water) to obtain a concentrated |
| | drug solution. |
| Step 2 | A volume of water or buffered solution with a fixed pH is placed in a first vessel and |
| | maintained at room temperature. |
| Step 3 | An aliquot of the concentrated drug solution is added to the contents of the first |
| | vessel to obtain a first sample solution having a desired target drug concentration. |
| | The drug concentration selected should be one which produces substantial |
| | precipitation and consequently higher apparent absorbance (i.e., turbidity) than a |
| | saturated solution having no such precipitation. |
| Step 4 | A test polymer is selected and, in a second vessel, the polymer is dissolved in |
| | water or a buffered solution with a fixed pH (identical in composition, pH and |
| | volume to that used in step C) in an amount sufficient to form a 0.25%-2% w/w |
| | polymer solution. |
| Step 5 | To form a second sample solution, an aliquot of the concentrated drug solution |
| | prepared in step A is added to the polymer solution in the second vessel to form a |
| | sample solution having a final drug concentration equal to that of the first sample |
| | solution. |
| Step 6 | At 60 minutes after preparation of both sample solutions, apparent absorbance |
| | (i.e., turbidity) of each sample solution is measured using light having a wavelength |
| | of 650 nm. |
| Step 7 | If the turbidity of the second sample solution is less than the turbidity of the first |
| | sample solution, the test polymer is deemed to be a “turbidity-decreasing polymer” |
| | and is useful as a crystallization inhibitor for the test drug. |

A technician performing Test I will readily find a suitable polymer concentration for the test within the polymer concentration range provided above, by routine experimentation. In a particularly preferred embodiment, a concentration of the polymer is selected such that when Test I is performed, the apparent absorbance of the second sample solution is not greater than about 50% of the apparent absorbance of the first sample solution.

Most surface stabilizers are described in detail in the Handbook of Pharmaceutical Excipients, published jointly by the American Pharmaceutical Association and The Pharmaceutical Society of Great Britain, the Pharmaceutical Press, 2000. The surface stabilizers are commercially available and/or can be prepared by techniques known in the art. Presentations of exemplary surfactants are given in McCutcheon, Detergents and Emulsifiers, Allied Publishing Co., New Jersey, 2004 and Van Os, Haak and Rupert, Physico-chemical Properties of Selected Anionic, Cationic and Nonionic Surfactants, Elsevier, Amsterdam, 1993.

The terms “pKa” and “Dissociation Constant” refer to a measure of the strength of an acid or a base. The pKa allows the determination of the charge on a molecule at any given pH.

The terms “logP” and “Partition Coefficient” refer to a measure of how well a substance partitions between a lipid (oil) and water. The Partition Coefficient is also a very useful parameter which may be used in combination with the pKa to predict the distribution of a compound in a biological system. Factors such as absorption, excretion and penetration of the CNS may be related to the Log P value of a compound and in certain cases predictions made.

The term “low aqueous solubility” refers to a therapeutic or diagnostic agent with a solubility in water of less than about 10 mg/mL. In another embodiment, the solubility in water is less than about 1 mg/mL.

The term “particle size” refers to effective diameter, in the longest dimension, of compound particles. Particle size is believed to be an important parameter affecting the clinical effectiveness of therapeutic or diagnostic agents of low aqueous solubility.

The terms “average particle size” and “mean particle size” refer to compound particle size of which at least 50% or more of the compound particles are, when measured by dynamic light scattering. In an exemplary embodiment, an average particle size of from about 120 nm to about 400 nm means that at least 50% of the compound particles have a particle size from about 120 nm to about 400 nm when measured by standard techniques, as indicated in other embodiments herein. In another embodiment, at least 70% of the particles, by weight, have a particle size of less than the indicated size. In another embodiment, at least 90% of the particles have the defined particle size. In yet another embodiment, at least 95% of the particles have the defined particle size. In another embodiment, at least 99% of the particles have the defined particle size. In other embodiments, different measurement techniques may be employed—such as laser diffraction.

### B. FORMULATIONS

The present invention comprises, in part, novel injectable depot formulations of low solubility drugs. The present invention also comprises a method of treating disorders suitable for treatment with low solubility drugs.

In one embodiment of the invention, an injectable depot formulation comprises: a) a pharmaceutically effective amount of a compound selected from the group consisting of a low solubility drug and a pharmaceutically acceptable salt thereof, the compound in the form of nanoparticles having an average particle size of less than about 2000 nm; b) a pharmaceutically acceptable carrier; and c) at least two surface stabilizers; wherein at least one of the surface stabilizers is adsorbed on the surface of the nanoparticles; and wherein the combined amount of the surface stabilizers is effective to maintain the average particle size of the nanoparticles (Formulation 1).

Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Suitable base salts are formed from bases which form non-toxic salts. Examples include the aluminum, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2002).

The compound may also exist in unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

Pharmaceutically acceptable salts of a low solubility drug may be prepared by one or more of three methods:

(i) by reacting the compound of formula I with the desired acid or base;

(ii) by removing an acid- or base-labile protecting group from a suitable precursor of the compound of formula I or by ring-opening a suitable cyclic precursor, for example, a lactone or lactam, using the desired acid or base; or

(iii) by converting one salt of a low solubility drug to another by reaction with an appropriate acid or base or by means of a suitable ion exchange column. All three reactions are typically carried out in solution. The resulting salt may precipitate out and be collected by filtration or may be recovered by evaporation of the solvent. The degree of ionization in the resulting salt may vary from completely ionized to almost non-ionized.

In another embodiment of the compound, the compound is crystalline.

In another embodiment of the injectable depot formulation, the pharmaceutically acceptable carrier is water.

In another embodiment of the injectable depot formulation, the nanoparticles of the compound have an average particle size of less than about 1500 nm. In still another embodiment, the nanoparticles have an average particle size of less than about 1000 nm. In still another embodiment, the nanoparticles have an average particle size of less than about 500 nm. In still another embodiment, the nanoparticles have an average particle size of less than about 350 nm.

In still another embodiment of the injectable depot formulation, the nanoparticles have an average particle size from about 120 nm to about 400 nm. In still another embodiment, the nanoparticles have an average particle size from about 220 nm to about 350 nm.

In another embodiment of the injectable depot formulation, the nanoparticles have an average particle size of about 250 nm.

In still another embodiment, nanoparticles have an average particle size of about 120 nm.

In still another embodiment, the nanoparticles have an average particle size of about 400 nm. In another embodiment of Formulation 1, the amount by weight of the compound is less than about 60% by weight of the total volume of the formulation. In still another embodiment, the amount by weight of the compound is less than about 40% by weight of the total volume of the formulation.

In another embodiment of Formulation 1, the amount by weight of the compound is at least about 1% by weight of the total volume of the formulation. In still another embodiment, the amount by weight of the compound is at least about 3% by weight of the total volume of the formulation. In still another embodiment, the amount by weight of the compound is at least about 15% by weight of the total volume of the formulation. In still another embodiment, the amount by weight of the compound is at least about 20% by weight of the total volume of the formulation In still another embodiment, the amount by weight of the compound is at least about 40% by weight of the total volume of the formulation.

In another embodiment of Formulation 1, the amount by weight of the compound is from about 1% by weight to about 60% by weight of the total volume of the formulation. In still another embodiment, the amount by weight of the compound is from about 3% by weight to about 60% by weight of the total volume of the formulation. In still another embodiment, the amount by weight of the compound is from about 15% by weight to about 60% by weight of the total volume of the formulation. In still another embodiment, the amount by weight of the compound is from about 20% by weight to about 60% by weight of the total volume of the formulation. In still another embodiment, the amount by weight of the compound is from about 1% by weight to about 40% by weight of the total volume of the formulation. In still another embodiment, wherein the amount by weight of the compound is from about 3% by weight to about 40% by weight of the total volume of the formulation. In still another embodiment, the amount by weight of the compound is from about 15% by weight to about 40% by weight of the total volume of the formulation. In still another embodiment, the amount by weight of the compound is from about 20% by weight to about 40% by weight of the total volume of the formulation.

In another embodiment of Formulation 1, a first surface stabilizer is a surfactant. In another embodiment of Formulation 1, a first surface stabilizer is selected from the group consisting of anionic surfactants, cationic surfactants, amphoteric surfactants, non-ionic surfactants and polymeric surfactants.

In another embodiment of Formulation 1, a first surface stabilizer is an anionic surfactant. In another embodiment, a first surface stabilizer is a cationic surfactant. In another embodiment, a first surface stabilizer is an amphoteric surfactant. In another embodiment, a first surface stabilizer is a non-ionic surfactant. In another embodiment, a first surface stabilizer is a polymeric surfactant.

In another embodiment of Formulation 1, a second surface stabilizer is selected from the group consisting of anionic surfactants, cationic surfactants, amphoteric surfactants, non-ionic surfactants and polymeric surfactants.

In another embodiment of Formulation 1, a second surface stabilizer is an anionic surfactant. In another embodiment, a second surface stabilizer is a cationic surfactant. In another embodiment, a second surface stabilizer is an amphoteric surfactant. In another embodiment, a second surface stabilizer is a non-ionic surfactant. In another embodiment, a second surface stabilizer is a polymeric surfactant.

In another embodiment of Formulation 1, a first surface stabilizer and a second surface stabilizer are independently selected from the group consisting of anionic surfactants, cationic surfactants, amphoteric surfactants, non-ionic surfactants and polymeric surfactants.

In another embodiment of Formulation 1, a first surface stabilizer is an anionic surfactant and a second surface stabilizer is an anionic surfactant. In yet another embodiment, a first surface stabilizer is an anionic surfactant and a second surface stabilizer is a cationic surfactant. In yet another embodiment, a first surface stabilizer is an anionic surfactant and a second surface stabilizer is an amphoteric surfactant. In yet another embodiment, a first surface stabilizer is an anionic surfactant and a second surface stabilizer is a non-ionic surfactant. In yet another embodiment, a first surface stabilizer is an anionic surfactant and a second surface stabilizer is a polymeric surfactant.

In another embodiment of Formulation 1, a first surface stabilizer is a cationic surfactant and a second surface stabilizer is an anionic surfactant. In yet another embodiment, a first surface stabilizer is an cationic surfactant and a second surface stabilizer is a cationic surfactant. In yet another embodiment, a first surface stabilizer is a cationic surfactant and a second surface stabilizer is an amphoteric surfactant. In yet another embodiment, a first surface stabilizer is a cationic surfactant and a second surface stabilizer is a non-ionic surfactant. In yet another embodiment, a first surface stabilizer is a cationic surfactant and a second surface stabilizer is a polymeric surfactant.

In another embodiment of Formulation 1, a first surface stabilizer is an amphoteric surfactant and a second surface stabilizer is an anionic surfactant. In yet another embodiment, a first surface stabilizer is an amphoteric surfactant and a second surface stabilizer is a cationic surfactant. In yet another embodiment, a first surface stabilizer is an amphoteric surfactant and a second surface stabilizer is an amphoteric surfactant. In yet another embodiment, a first surface stabilizer is an amphoteric surfactant and a second surface stabilizer is a non-ionic surfactant. In yet another embodiment, a first surface stabilizer is an amphoteric surfactant and a second surface stabilizer is a polymeric surfactant.

In another embodiment of Formulation 1, a first surface stabilizer is a non-ionic surfactant and a second surface stabilizer is an anionic surfactant. In yet another embodiment, a first surface stabilizer is a non-ionic surfactant and a second surface stabilizer is a cationic surfactant. In yet another embodiment, a first surface stabilizer is a non-ionic surfactant and a second surface stabilizer is am amphoteric surfactant. In yet another embodiment, a first surface stabilizer is a non-ionic surfactant and a second surface stabilizer is a non-ionic surfactant. In yet another embodiment, a first surface stabilizer is a non-ionic surfactant and a second surface stabilizer is a polymeric surfactant.

In another embodiment of Formulation 1, a first surface stabilizer is a polymeric surfactant and a second surface stabilizer is an anionic surfactant. In yet another embodiment, a first surface stabilizer is a polymeric surfactant and a second surface stabilizer is a cationic surfactant. In yet another embodiment, a first surface stabilizer is a polymeric surfactant and a second surface stabilizer is an amphoteric surfactant. In yet another embodiment, a first surface stabilizer is a polymeric surfactant and a second surface stabilizer is a non-ionic surfactant. In yet another embodiment, a first surface stabilizer is a polymeric surfactant and a second surface stabilizer is a polymeric surfactant.

In another embodiment of Formulation 1, a first surface stabilizer is selected from the group consisting of Pluronic® F108 and Tween® 80 and a second surface stabilizer is selected from the group consisting of Pluronic® F108, Tween® 80, and SLS. In another embodiment of Formulation 1, a first surface stabilizer is PVP and a second surface stabilizer is Pluronic® F108. In another embodiment a first surface stabilizer is PVP and a second surface stabilizer is Pluronic® F68. In another embodiment, a first surface stabilizer is PVP and a second surface stabilizer is SLS. In another embodiment, a first surface stabilizer is Pluronic® F108 and a second surface stabilizer is Tween® 80. In another embodiment, a first surface stabilizer is PVP and a second surface stabilizer is Tween® 80.

In another embodiment of Formulation 1, the amount by weight of a first surface stabilizer is from about 0.5% to about 3.0% by weight of the total volume of the formulation. In another embodiment, the amount by weight of a first surface stabilizer is from about 0.5% to about 2.0% by weight of the total volume of the formulation. In yet another embodiment of Formulation 1, the amount by weight of a first surface stabilizer is about 0.5% by weight of the total volume of the formulation. In yet another embodiment of Formulation 1, the amount by weight of a first surface stabilizer is about 1.0% by weight of the total volume of the formulation. In yet another embodiment of Formulation 1, the amount by weight of a first surface stabilizer is about 2.0% by weight of the total volume of the formulation.

In an embodiment of Formulation 1, the amount by weight of a second surface stabilizer is from about 0.1% to about 3.0%, preferably about 0.1% to about 2.0%, by weight of the total volume of the formulation. In another embodiment of Formulation 1, the amount by weight of a second surface stabilizer is from about 0.1% to about 1.0% by weight of the total volume of the formulation. In another embodiment of Formulation 1, the amount by weight of a second surface stabilizer is about 2.0% by weight of the total volume of the formulation. In still another embodiment of Formulation 1, the amount by weight of a second surface stabilizer is about 0.5% by weight of the total volume of the formulation. In still another embodiment of Formulation 1, the amount by weight of a second surface stabilizer is about 0.1% by weight of the total volume of the formulation.

In an embodiment of Formulation 1, a third surface stabilizer is present, wherein the amount by weight of the third surface stabilizer is from about 0.018% to about 1.0% by weight of the total volume of the formulation. In another embodiment of Formulation 1, the amount by weight of the third surface stabilizer is about 0.018% by weight of the total volume of the formulation. In still another embodiment, the amount by weight of the third surface stabilizer is about 0.1% by weight of the total volume of the formulation. In still another embodiment, the amount by weight of the third surface stabilizer is about 0.02% by weight of the total volume of the formulation. In still another embodiment, the amount by weight of the third surface stabilizer is about 0.5% by weight of the total volume of the formulation. In still another embodiment, the amount by weight of the third surface stabilizer is about 1.0% by weight of the total volume of the formulation.

In another embodiment of Formulation 1, a third surface stabilizer is a surfactant. In another embodiment, the third surfactant is selected from the group consisting of Pluronic® F68, benzalkonium chloride, lecithin and SLS. In another embodiment, a third surface stabilizer is Pluronic® F68. In another embodiment, a third surface stabilizer is benzalkonium chloride. In another embodiment, a third surface stabilizer is lecithin. In another embodiment, a third surface stabilizer is SLS.

In another embodiment of the invention, the total amount by weight of surface stabilizers in a formulation is about 6% or less, more preferably about 5% or less.

In an embodiment of Formulation 1, a bulking agent is present, wherein the amount by weight of the third surface stabilizer is from about 1.0% to about 10.0% by weight of the total volume of the formulation. In another embodiment of Formulation 1, the amount by weight of the bulking agent is about 1.0% by weight of the total volume of the formulation. In another embodiment, the amount by weight of the third surface stabilizer is about 5.0% by weight of the total volume of the formulation. In another embodiment, the amount by weight of the third surface stabilizer is about 10.0% by weight of the total volume of the formulation.

In another embodiment of Formulation 1, a bulking agent is present, the bulking agent selected from the group consisting of trehalose, mannitol and PEG400. In another embodiment, the bulking agent is trehalose. In another embodiment, the bulking agent is mannitol. In another embodiment, the bulking agent is PEG400.

In another embodiment of Formulation 1, the formulation consists essentially of a compound, a carrier, a first surface stabilizer and a second surface stabilizer, as previously defined herein. In another embodiment, the formulation consists essentially of a compound, a carrier, a first surface stabilizer, a second surface stabilizer and a third surface stabilizer, as previously defined herein. In yet another embodiment, the formulation consists essentially of a compound, a carrier, a first surface stabilizer, a second surface stabilizer and a bulking agent, as previously defined herein. These variations are summarized in the following table:

TABLE B-2

| parameter | Formulation 2 | Formulation 3 | Formulation 4 |
|---|---|---|---|
| first surface stabilizer | Yes | Yes | Yes |
| second surface | Yes | Yes | Yes |
| stabilizer | | | |
| third surface | No | Yes | No |
| stabilizer | | | |
| bulking agent | No | No | Yes |
| Crystalline | Yes | Yes | Yes |
| Compound? | | | |

### C. METHODS OF PREPARATION AND TREATMENT

The compound nanoparticles can be made using several different methods, including, for example, milling, precipitation and high pressure homogenization. Exemplary methods of making compound nanoparticles are described in U.S. Pat. No. 5,145,684, the entire content of which is incorporated by reference herein. The optimal effective average particle size of the invention can be obtained by controlling the process of particle size reduction, such as controlling the milling time and the amount of surface stabilizer added. Crystal growth and particle aggregation can also be minimized by milling or precipitating the composition under colder temperatures, and by storing the final composition at colder temperatures.

1. Aqueous Milling

In one embodiment of the invention, there is provided a method of preparing the injectable depot formulation of the compound according to Formulation 1. Milling of compound in aqueous solution to obtain a nanoparticulate dispersion comprises dispersing compound in water, followed by applying mechanical means in the presence of grinding media to reduce the particle size of the compound to the desired effective average particle size, the optimal sizes as provided in other embodiments herein. The compound can be effectively reduced in size in the presence of two or more surface stabilizers. Alternatively, the compound can be contacted with two or more surface stabilizers after attrition. Other compounds, such as a bulking agent, can be added to the compound/surface stabilizer mixture during the size reduction process. Dispersions can be manufactured continuously or in a batch mode. The resultant nanoparticulate drug dispersion can be utilized in solid or liquid dosage formulations. In another embodiment, the nanoparticulate dispersion may be utilized in intramuscular depot formulations suitable for injection.

Exemplary useful mills include low energy mills, such as a roller mill, attritor mill, vibratory mill and ball mill, and high energy mills, such as Dyno mills, Netzsch mills, DC mills, and Planetary mills. Media mills include sand ills and bead mills. In media milling, the compound is placed into a reservoir along with a dispersion medium (for example, water) and at least two surface stabilizers. The mixture is recirculated through a chamber containing media and a rotating shaft/impeller. The rotating shaft agitates the media which subjects the compound to impacting and sheer forces, thereby reducing particle size.

2. Grinding Media

Exemplary grinding media comprises particles that are substantially spherical in shape, such as beads, consisting essentially of polymeric resin. In another embodiment, the grinding media comprises a core having a coating of a polymeric resin adhered thereon. Other examples of grinding media comprise essentially spherical particles comprising glass, metal oxide, or ceramic.

In general, suitable polymeric resins are chemically and physically inert, substantially free of metals, solvent, and monomers, and of sufficient hardness and friability to enable them to avoid being chipped or crushed during grinding. Suitable polymeric resins include, without limitation: crosslinked polystyrenes, such as polystyrene crosslinked with divinylbenzene; styrene copolymers; polycarbonates; polyacetals, for example, Delrin® (E.I. du Pont de Nemours and Co.); vinyl chloride polymers and copolymers; polyurethanes; polyamides; poly(tetrafluoroethylenes), for example, Teflon® (E.I. du Pont de Nemours and Co.), and other fluoropolymers; high density polyethylenes; polypropylenes; cellulose ethers and esters such as cellulose acetate; polyhydroxymethacrylate; polyhydroxyethyl acrylate; and silicone-containing polymers such as polysiloxanes. The polymer can be biodegradable. Exemplary biodegradable polymers include poly(lactides), poly(glycolide) copolymers of lactides and glycolide, polyanhydrides, poly(hydroxyethyl methacylate), poly(imino carbonates), poly(N-acylhydroxyproline)esters, poly(N-palmitoyl hydroxyproline) esters, ethylene-vinyl acetate copolymers, poly(orthoesters), poly(caprolactones), and poly(phosphazenes). For biodegradable polymers, contamination from the media itself advantageously can metabolize in vivo into biologically acceptable products that can be eliminated from the body.

The grinding media preferably ranges in size from about 10 μm to about 3 mm. For fine grinding, exemplary grinding media is from about 20 μm to about 2 mm. In another embodiment, exemplary grinding media is from about 30 μm to about 1 mm in size. In another embodiment, the grinding media is about 500 μm in size. The polymeric resin can have a density from about 0.8 to about 3.0 g/ml.

In one exemplary grinding process, the particles are made continuously. Such a method comprises continuously introducing compound into a milling chamber, contacting the compound with grinding media while in the chamber to reduce the compound particle size, and continuously removing the nanoparticulate compound from the milling chamber.

The grinding media is separated from the milled nanoparticulate compound using conventional separation techniques in a secondary process, including, without limitation, simple filtration, sieving through a mesh filter or screen, and the like. Other separation techniques such as centrifugation may also be employed.

3. Precipitation

Another method of forming the desired nanoparticulate dispersion is by microprecipitation. This is a method of preparing stable dispersions of drugs in the presence of two or more surface stabilizers and one or more colloid stability enhancing surface active agents free of any trace toxic solvents or solubilized heavy metal impurities. An exemplary method comprises: (1) dissolving the compound in a suitable solvent; (2) adding the formulation from step (1) to a solution comprising at least two surface stabilizers to form a clear solution; and (3) precipitating the formulation from step (2) using an appropriate non-solvent. The method can be followed by removal of any formed salt, if present, by dialysis or diafiltration and concentration of the dispersion by conventional means. The resultant nanoparticulate drug dispersion can be utilized in solid or liquid dosage formulations. In another embodiment, the nanoparticulate dispersion may be utilized in intramuscular depot formulations suitable for injection.

4. Homogenization

Another method of forming the desired nanoparticulate dispersion is by homogenization. Like precipitation, this technique does not use milling media. Instead, compound, surface stabilizers and carrier—the “mixture” (or, in an alternative embodiment, compound and carrier with the surface stabilizers added following reduction in particle size) constitute a process stream propelled into a process zone, which in a Microfluidizer® (Microfluidics Corp.) is called the Interaction Chamber. The mixture to be treated is inducted into the pump and then forced out. The priming valve of the Microfluidizer® purges air out of the pump. Once the pump is filled with the mixture, the priming valve is closed and the mixture is forced through the Interaction Chamber. The geometry of the Interaction Chamber produces powerful forces of sheer, impact and cavitation which reduce particle size. Inside the Interaction Chamber, the pressurized mixture is split into two streams and accelerated to extremely high velocities. The formed jets are then directed toward each other and collide in the interaction zone. The resulting product has very fine and uniform particle size.

5. Sterile Product Manufacturing

Development of injectable compositions requires the production of a sterile product. The manufacturing process of the present invention is similar to typical known manufacturing processes for sterile suspensions. A typical sterile suspension manufacturing process flowchart is as follows:

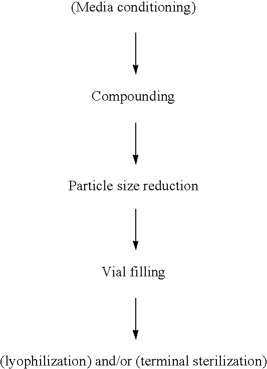

As indicated by the optional steps in parentheses, some of the processing is dependent upon the method of particle size reduction and/or method of sterilization. For example, media conditioning is not required for a milling method that does not use media. If terminal sterilization is not feasible due to chemical and/or physical instability, aseptic processing can be used. Terminal sterilization can be by steam sterilization or by high energy irradiation of the product.

6. Methods of Treatment

Conditions

The conditions that can be treated in accordance with the present invention include one or more disorders selected from the group consisting of: arthritis, including rheumatoid arthritis, spondyloarthropathies, gouty arthritis, osteoarthritis, systemic lupus erythematosus and juvenile arthritis, osteoarthritis, and other arthritic conditions; central nervous system disorders (including, but not limited to, central nervous system disorders having an inflammatory or apoptotic component), such as Alzheimer's disease, Parkinson's disease, Huntington's Disease, amyotrophic lateral sclerosis, spinal cord injury, and peripheral neuropathy; peripheral nervous system disorders; cardiovascular diseases including atherosclerosis, myocardial infarction (including post-myocardial infarction indications), thrombosis, congestive heart failure, and cardiac reperfusion injury, as well as complications associated with hypertension and/or heart failure such as vascular organ damage, restenosis; gastrointestinal conditions such as inflammatory bowel disease, Crohn's disease, gastritis, irritable bowel syndrome and ulcerative colitis, parasitic infections, microbial diseases, neoplastic disorders, immunological disorders, blood disorders, hormone deficiencies, skin-related conditions such as psoriasis, eczema, burns, dermatitis, keloid formation, scar tissue formation, and angiogenic disorders; opthalmological conditions such as corneal graft rejection, ocular neovascularization, retinal neovascularization including neovascularization following injury or infection, and retrolental fibroplasia; glaucoma including primary open angle glaucoma (POAG), juvenile onset primary open-angle glaucoma, angle-closure glaucoma, pseudoexfoliative glaucoma, anterior ischemic optic neuropathy (AION), ocular hypertension, Reiger's syndrome, normal tension glaucoma, neovascular glaucoma, ocular inflammation and corticosteroid-induced glaucoma; acute injury to the eye tissue and ocular traumas such as post-traumatic glaucoma, traumatic optic neuropathy, and central retinal artery occlusion (CRAO); ophthalmic diseases such as retinitis, retinopathies (including diabetic retinopathy), uveitis, ocular photophobia, nonglaucomatous optic nerve atrophy, and age related macular degeneration (ARMD) (including ARMD-atrophic form) and pathological, but non-malignant, conditions such as hemaginomas, including infantile hemaginomas, angiofibroma of the nasopharynx and avascular necrosis of bone; benign and malignant tumors/neoplasia including cancer, such as colorectal cancer, brain cancer, bone cancer, epithelial cell-derived neoplasia (epithelial carcinoma) such as basal cell carcinoma, adenocarcinoma, gastrointestinal cancer such as lip cancer, mouth cancer, esophageal cancer, small bowel cancer and stomach cancer, colon cancer, liver cancer, bladder cancer, pancreas cancer, ovarian cancer, cervical cancer, lung cancer, breast cancer and skin cancer, such as squamous cell and basal cell cancers, prostate cancer, renal cell carcinoma, and other known cancers that affect epithelial cells throughout the body; leukemia; lymphoma; systemic lupus erthrematosis (SLE); angiogenesis including neoplasia; and metastasis; inflammation; neuroinflammation; allergy; neuropathic pain; fever; pulmonary disorders or lung inflammation, including adult respiratory distress syndrome, pulmonary sarcoidosis, asthma, silicosis, chronic pulmonary inflammatory disease, chronic obstructive pulmonary disease (COPD), and asthma; cardiomyopathy; stroke including ischemic and hemorrhagic stroke; ischemia including brain ischemia and ischemia resulting from cardiac/coronary bypass; reperfusion injury; renal reperfusion injury; brain edema; neurotrauma and brain trauma including closed head injury; neurodegenerative disorders; liver disease and nephritis; ulcerative diseases such as gastric ulcer; periodontal disease; diabetes; diabetic nephropathy; viral and bacterial infections, including sepsis, septic shock, gram negative sepsis, malaria, meningitis, HIV infection, opportunistic infections, cachexia secondary to infection or malignancy, cachexia secondary to acquired immune deficiency syndrome (AIDS), AIDS, ARC (AIDS related complex), pneumonia, and herpes virus; myalgias due to infection; influenza; endotoxic shock and sepsis; toxic shock syndrome; autoimmune disease including graft vs. host reaction and allograft rejections; treatment of bone resorption diseases, such as osteoporosis; multiple sclerosis; and disorders of the female reproductive system such as endometriosis.

Administration and Dosing

Typically, a formulation described in this specification is administered in an amount effective to treat conditions listed herein. The depot formulations of the present invention are administered by injection, whether subcutaneously or intramuscularly, and in a dose effective for the treatment intended. Therapeutically effective doses of the compounds required to prevent or arrest the progress of or to treat the medical condition are readily ascertained by one of ordinary skill in the art using preclinical and clinical approaches familiar to the medicinal arts.

An effective dose for injection of a formulation of the invention can be generally determined by a physician of ordinary skill in the art. The effective dose can be determined taking into consideration factors know to those of skill in the art, such as the indication being treated, the weight of the patient, and the duration of treatment (e.g. days or weeks) desired. The percentage of drug present in the formulation is also a factor. An example of an effective dose for injection of a formulation of the present invention is from about 0.1 ml to about 2.5 ml injected once every 1, 2, 3 or 4 weeks. Preferably, the dose for injection is about 2 ml or less, for example from about 1 ml to about 2 ml.

7. Use in the Preparation of a Medicament

In one embodiment, the present invention comprises methods for the preparation of a formulation (or “medicament”) comprising the Formulations of other embodiments herein disclosed, in combination with one or more pharmaceutically-acceptable carriers and at least two surface stabilizers, wherein at least one surface stabilizer is adsorbed on to the surface of the compound nanoparticles and wherein the combined amount of the surface stabilizers is effective to maintain the average particle size of the nanoparticles (Formulation 1), in which such formulation is suitable in treating one or more conditions selected from the group consisting of: The conditions that can be treated in accordance with the present invention include one or more disorders selected from the group consisting of: arthritis, including rheumatoid arthritis, spondyloarthropathies, gouty arthritis, osteoarthritis, systemic lupus erythematosus and juvenile arthritis, osteoarthritis, and other arthritic conditions; central nervous system disorders (including, but not limited to, central nervous system disorders having an inflammatory or apoptotic component), such as Alzheimer's disease, Parkinson's disease, Huntington's Disease, amyotrophic lateral sclerosis, spinal cord injury, and peripheral neuropathy; peripheral nervous system disorders; cardiovascular diseases including atherosclerosis, myocardial infarction (including post-myocardial infarction indications), thrombosis, congestive heart failure, and cardiac reperfusion injury, as well as complications associated with hypertension and/or heart failure such as vascular organ damage, restenosis; gastrointestinal conditions such as inflammatory bowel disease, Crohn's disease, gastritis, irritable bowel syndrome and ulcerative colitis, parasitic infections, microbial diseases, neoplastic disorders, immunological disorders, blood disorders, hormone deficiencies, skin-related conditions such as psoriasis, eczema, burns, dermatitis, keloid formation, scar tissue formation, and angiogenic disorders; opthalmological conditions such as corneal graft rejection, ocular neovascularization, retinal neovascularization including neovascularization following injury or infection, and retrolental fibroplasia; glaucoma including primary open angle glaucoma (POAG), juvenile onset primary open-angle glaucoma, angle-closure glaucoma, pseudoexfoliative glaucoma, anterior ischemic optic neuropathy (AION), ocular hypertension, Reiger's syndrome, normal tension glaucoma, neovascular glaucoma, ocular inflammation and corticosteroid-induced glaucoma; acute injury to the eye tissue and ocular traumas such as post-traumatic glaucoma, traumatic optic neuropathy, and central retinal artery occlusion (CRAO); ophthalmic diseases such as retinitis, retinopathies (including diabetic retinopathy), uveitis, ocular photophobia, nonglaucomatous optic nerve atrophy, and age related macular degeneration (ARMD) (including ARMD-atrophic form) and pathological, but non-malignant, conditions such as hemaginomas, including infantile hemaginomas, angiofibroma of the nasopharynx and avascular necrosis of bone; benign and malignant tumors/neoplasia including cancer, such as colorectal cancer, brain cancer, bone cancer, epithelial cell-derived neoplasia (epithelial carcinoma) such as basal cell carcinoma, adenocarcinoma, gastrointestinal cancer such as lip cancer, mouth cancer, esophageal cancer, small bowel cancer and stomach cancer, colon cancer, liver cancer, bladder cancer, pancreas cancer, ovarian cancer, cervical cancer, lung cancer, breast cancer and skin cancer, such as squamous cell and basal cell cancers, prostate cancer, renal cell carcinoma, and other known cancers that affect epithelial cells throughout the body; leukemia; lymphoma; systemic lupus erthrematosis (SLE); angiogenesis including neoplasia; and metastasis; inflammation; neuroinflammation; allergy; neuropathic pain; fever; pulmonary disorders or lung inflammation, including adult respiratory distress syndrome, pulmonary sarcoidosis, asthma, silicosis, chronic pulmonary inflammatory disease, chronic obstructive pulmonary disease (COPD), and asthma; cardiomyopathy; stroke including ischemic and hemorrhagic stroke; ischemia including brain ischemia and ischemia resulting from cardiac/coronary bypass; reperfusion injury; renal reperfusion injury; brain edema; neurotrauma and brain trauma including closed head injury; neurodegenerative disorders; liver disease and nephritis; ulcerative diseases such as gastric ulcer; periodontal disease; diabetes; diabetic nephropathy; viral and bacterial infections, including sepsis, septic shock, gram negative sepsis, malaria, meningitis, HIV infection, opportunistic infections, cachexia secondary to infection or malignancy, cachexia secondary to acquired immune deficiency syndrome (AIDS), AIDS, ARC (AIDS related complex), pneumonia, and herpes virus; myalgias due to infection; influenza; endotoxic shock and sepsis; toxic shock syndrome; autoimmune disease including graft vs. host reaction and allograft rejections; treatment of bone resorption diseases, such as osteoporosis; multiple sclerosis; and disorders of the female reproductive system such as endometriosis.

### D. WORKING EXAMPLES

The following examples illustrate the present invention. Additional embodiments of the present invention may be prepared using information presented in these Working Examples, either alone or in combination with techniques generally known in the art. In these working examples, percentages, when given to describe components of the formulation, are in the unit weight per volume, or w/v.

### Example 1

### Preparation of Formulation A

A coarse suspension was prepared by placing 8.86 gm of ziprasidone free base in a 100 ml milling chamber with 48.90 gm of milling media (500 micron polystyrene beads). To this, 4.2 ml each of 10% solutions of Pluronic® F108 and Tween® 80 were added. In addition, 27.8 ml of water for injection was added to the milling chamber. The above mixture was stirred until uniform suspension was obtained. This suspension was then milled for 30 minutes at 2100 RPM in a Nanomill-1 (Manufacturer Elan Drug Delivery, Inc.) and the temperature during milling was maintained at 4° C. The resulting suspension was filtered under vacuum to remove the milling media and the suspension characterized by microscopy and light scattering (Brookhaven). For microscopic observation, a drop of diluted suspension was placed between a cover slip and slide and observed under both bright and dark field. For particle size determination by light scattering, a drop of suspension was added to a sample cuvette filled with water and particle size measured. The reported values are effective diameter in nm.

The above suspension after milling was free flowing and did not show any large crystals under the microscope at 400× and dispersed particles could not be seen individually due to the rapid Brownian motion. The effective diameter of the 21% ziprasidone free base nanosuspension was 235 nm.

### Example 2

### Preparation of Formulation B

A coarse suspension was prepared by placing 8.84 gm of ziprasidone free base in a 100 ml milling chamber with 48.90 gm of milling media (500 micron polystyrene beads). To this, 4.2 ml of 10% solution of Pluronic® F108 was added. In addition, 32 ml of water for injection was added to the milling chamber. The above mixture was milled under identical conditions as in example 1.

When the milling was stopped at 30 minutes, the above suspension turned into a paste and thus a uniform non-aggregated free flowing nanosuspension was not obtained. Since the paste could not be filtered to separate the milling media, additional characterization could not be performed.

### Example 3

### Preparation of Formulation C

A coarse suspension was prepared by placing 8.82 gm of ziprasidone free base in the 100 ml milling chamber with 48.87 gm of milling media (500 micron polystyrene beads).

To this, 4.2 ml of 10% solution of PVP-K30 was added. In addition, 32 ml of water for injection was added to the milling chamber. The above mixture was milled under identical conditions as in example 1.

When the milling was stopped at 30 minutes, the above suspension turned into a paste and thus a uniform non-aggregated free flowing nanosuspension was not obtained. Since the paste could not be filtered to separate the milling media, additional characterization could not be performed.

### Example 4

### Preparation of Formulation D

A 21% ziprasidone free base coarse suspension was prepared in 2.5% aqueous solution of Pluronic® F108. This suspension was diluted 1:1 v/v with water to result in 10.5% ziprasidone free base suspension with 1.25% of Pluronic® F108 in water. The suspension was milled in a 100 ml milling chamber with milling media (500 micron polystyrene beads) at 5500 RPM.

When the milling was stopped at 1 hour, the above suspension after filtration was free flowing and did not show any large crystals under the microscope and the rapid Brownian motion was observed of the particles. The effective diameter of the 10.5% ziprasidone free base nanosuspension was 181 nm.

### Example 5

### Preparation of Formulation E

A coarse suspension was prepared by placing 9.69 gm of ziprasidone hydrochloride in a 100 ml milling chamber with 48.96 gm of milling media (500 micron polystyrene beads). To this, 4.2 ml each of the 10% PVP and 10% of Pluronic® F108 solutions were added. In addition, 25.4 ml of water for injection was added to the milling chamber. The above mixture was milled under identical conditions for 3 hours as in example 1.

When the milling was stopped at 3 hours, the above suspension after filtration was free flowing and did not show any large crystals under the microscope and the rapid Brownian motion was observed of the particles. The effective diameter of the 23% ziprasidone hydrochloride nanosuspension was 117 nm.

### Example 6

### Preparation of Formulation F

A coarse suspension was prepared by placing 11.78 gm of ziprasidone mesylate in a 100 ml milling chamber with 48.89 gm of milling media (500 micron polystyrene beads). To this, 8.4 ml of 10% PVP and 2.1 ml of 10% of Pluronic® F108 solutions were added. In addition, 24.2 ml of water for injection was added to the milling chamber. The above mixture was milled under identical conditions for 3 hours as in example 1.

When the milling was stopped at 3 hours, the above suspension after filtration was free flowing and did not show any large crystals under the microscope and the rapid Brownian motion was observed of the particles. The effective diameter of the 28% ziprasidone mesylate nanosuspension was 406 nm.

### Example 7

### Preparation of Formulation G

A coarse suspension was prepared by placing 8.85 gm of ziprasidone free base in the 100 ml milling chamber with 48.89 gm of milling media (500 micron polystyrene beads). To this, 4.2 ml each of 10% solutions of Pluronic® F108, Tween® 80 and 5% Lecithin solutions were added. In addition, 23.8 ml of water for injection was added to the milling chamber. The above mixture was stirred until uniform suspension was obtained. This suspension was then milled for 30 minutes at 2100 RPM in a Nanomill-1 (Manufacturer Elan Drug Delivery, Inc.) and the temperature during milling was maintained at 4° C. The resulting suspension was filtered under vacuum to remove the milling media and the suspension characterized by microscopy and light scattering as described in example 1.

### Example 8

### Preparation of Formulation Ft

A coarse suspension was prepared by placing 8.87 gm of ziprasidone free base in the 100 ml milling chamber with 48.9 gm of milling media (500 micron polystyrene beads). To this, 4.2 ml of 10% Tween® 80 solution and 8.4 ml of 10% Pluronic® F108 solution were added. In addition, 23.6 ml of water for injection was added to the milling chamber. The above mixture was stirred until uniform suspension was obtained. This suspension was then milled for 30 minutes at 2100 RPM in a Nanomill-1 (Manufacturer Elan Drug Delivery, Inc.) and the temperature during milling was maintained at 4° C. The resulting suspension was filtered under vacuum to remove the milling media and the suspension characterized by microscopy and light scattering as described in example 1.

### Example 9

### Stability of an Exemplary Formulation Comprising 21% Ziprasidone Free Base Nanoparticles

The particle size of Formulation A packaged in a vial stored at 5° C. was monitored. For particle size determination by light scattering a drop of suspension was added to a sample cuvette filled with water and particle size measured. The reported values are effective diameter in nm. The results are listed in D-1.

TABLE D-1
Effective Particle Diameter of Formulation A Stored at 5° C.

| Time (days) | Effective diameter (nm) |
|---|---|
| 0 | 233 |
| 5 | 230 |
| 50 | 233 |
| 60 | 238 |
| 92 | 234 |
| 130 | 245 |
| 220 | 246 |
| 339 | 248 |
| 700 | 256 |

### Example 10

### Stability of an Exemplary Formulation Comprising 23% Ziprasidone HCl Nanoparticles

The particle size of Formulation E packaged in a vial stored at 5° C. was monitored. For particle size determination by light scattering a drop of suspension was added to a sample cuvette filled with water and particle size measured. The reported values are effective diameter in nm. The results are listed in the following table.

TABLE D-2
Effective Particle Diameter of Formulation E Stored at 5° C.

| Time (days) | Effective diameter (nm) |
|---|---|
| 0 | 117 |
| 4 | 120 |
| 7 | 126 |
| 52 | 142 |
| 85 | 136 |
| 123 | 142 |

### Example 11

### Stability of an Exemplary Formulation Comprising 28% Ziprasidone Mesylate Nanoparticles

The particle size of Formulation F packaged in a vial stored at 5° C. was monitored. For particle size determination by light scattering a drop of suspension was added to a sample cuvette filled with water and particle size measured. The reported values are effective diameter in nm. The results are listed in the following table.

TABLE D-3
Effective Particle Diameter of Formulation F Stored at 5° C.

| Time (days) | Effective diameter (nm) |
|---|---|
| 0 | 406 |
| 5 | 444 |
| 50 | 415 |
| 60 | 407 |
| 92 | 518 |
| 130 | 485 |
| 339 | 525 |
| 700 | 609 |

### Example 12

Sterilization and Storage Stability of Formulation G

The filtered suspension of Example 7 was filled (3 ml) into flint vials. The vials were sealed with a rubber stopper and an aluminum seal was crimped on the stopper. The filled vials were sterilized for 15 min at 121° C. in a steam sterilizer. The suspension after sterilization was characterized and particle size measured by light scattering. The filled vials were stored at 5° C. and sampled at various times to determine particle size and stability of the suspension.

The following table shows particle size stability of Formulation G during autoclaving and upon storage of the sterilized formulation.

TABLE D-4
Effective Particle Diameter of Formulation G after
Autoclaving and upon Storage a 5° C.

| | Effective diameter (nm) |
|---|---|
| Time | |
| Before Sterilization | 235 nm |
| After Sterilization | 267 nm |
| Storage Time (days) | |
| post-sterilization | |
| 0 | 274 |
| 4 | 281 |
| 7 | 271 |
| 16 | 268 |
| 36 | 274 |

### Example 13

### Sterilization and Storage Stability of Formulation H

The filtered suspension of Example 8 was filled (3 ml) into flint vials. The vials were sealed with a rubber stopper and an aluminum seal was crimped on the stopper. The filled vials were sterilized for 15 min at 121° C. in a steam sterilizer. The suspension after sterilization was characterized and particle size measured by light scattering. The filled vials were stored at 5° C. and sampled at various times to determine particle size and stability of the suspension. The following table shows particle size stability of Formulation H during autoclaving and upon storage of the sterilized formulation.

TABLE D-5
Effective Particle Diameter of Formulation H after
Autoclaving and upon Storage at 5° C.

| | Effective diameter (nm) |
|---|---|
| Time | |
| Before Sterilization | 234 nm |
| After Sterilization | 311 nm |
| Storage Time (days) | |
| post-sterilization | |
| 0 | 319 |
| 3 | 331 |
| 6 | 325 |
| 15 | 313 |
| 35 | 319 |

### Example 14

### Stability of Ziprasidone Nanosuspensions: Monitoring of Particle Size Using Dynamic Light Scattering

It was surprisingly discovered that use of a single surface stabilizer was not sufficient to allow the suspension post-milling to resolve into a uniform free-flowing suspension without large crystals. Instead, as shown in Table D-6 and Working Examples 2 and 3, use of a single surface stabilizer resulted in only an unresolvable paste. However, when two or more surface stabilizers were present, a free flowing suspension resolved. Upon closer examination, the data shows that a smaller particle size (initial effective diameter) is achieved, even when the total volume of the two surfactants is less than the total volume of a single surfactant. Thus, similar compounds similar in nature to ziprasidone (low water solubility, logP about 3 or greater, e.g.) may also require two surface stabilizers to maintain particle size in a nanoparticle formulation.

Without being bound by theory, it may be that the combination of two or more surface stabilizers provide enhanced surface stability and improve the ability of the crystal to maintain its nanoparticulate size without aggregation. The addition of a different, second surface stabilizer may allow for the reduction in total amount of surface stabilizers by % w/v, which supports a synergistic interaction between surface stabilizers. That is, the use of at least two surface stabilizers in a formulation with a compound of low water solubility seems to maintain particle size and, therefore, provide a foundation for an IM depot formulation of interest.

TABLE D-6
Nanosuspensions of Ziprasidone and Particle Size

| | | | % | | | | |
|---|---|---|---|---|---|---|---|
| | % | % | Tween | other | milling | Time | Initial effective |
| Z - Com. | PVP | F108 | 80 | additives | time | (days) | diameter (nm) |
| 21% FB | 1 | | | | 30 min | 0 | — |
| 21% FB | 1 | 1 | | | 30 min | 0 | 242 |
| 21% FB | 1 | 1 | | | 30 min | 0 | 312 |
| 21% FB | 1 | 0.5 | | | 30 min | 0 | 309 |
| 21% FB | 1 | 1 | | | 10 min | 0 | 390 |
| 21% FB | 1 | 1 | | | 20 min | 0 | 255 |
| 21% FB | 1 | 1 | | | 30 min | 0 | 232 |
| 21% FB | 1 | 1 | | | 45 min | 0 | 234 |
| 21% FB | 1 | 1 | | | 30 min | 0 | 249 |
| 21% FB | 1 | 1 | | | 60 min | 0 | 230 |
| 21% FB | 1 | 1 | | | 60 min | 55 | 190 |
| 21% FB | 1 | 1 | | | 60 min | 0 | 252 |
| 21% FB | 1 | 1 | | | 60 min | 45 | 201 |
| 21% FB | 1 | 1 | | | 60 min | 52 | 231 |
| 21% FB | 1 | 1 | | | 60 min | 105 | 238 |
| 21% FB | 1 | 1 | | | 60 min | 143 | 261 |
| 21% FB | 1 | 1 | | | 60 min | 352 | 220 |
| 21% FB | | 1 | 1 | | 30 min | 0 | 234 |
| 21% FB | | 1 | | | 90 min | 0 | — |
| 21% FB | | 1 | | | 30 min | 0 | — |
| 21% FB | | 1 | 1 | | 30 min | 0 | 220 |
| 21% FB | | 2 | 1 | | 30 min | 0 | 234 |
| 21% FB | | 1 | 1 | | 30 min | 0 | 233 |
| 21% FB | | 1 | 1 | | 30 min | 5 | 230 |
| 21% FB | | 1 | 1 | | 30 min | 50 | 233 |
| 21% FB | | 1 | 1 | | 30 min | 60 | 238 |
| 21% FB | | 1 | 1 | | 30 min | 92 | 234 |
| 21% FB | | 1 | 1 | | 30 min | 130 | 245 |
| 21% FB | | 1 | 1 | | 30 min | 220 | 246 |
| 21% FB | | 1 | 1 | | 30 min | 339 | 248 |
| 21% FB | | 1 | 1 | | 30 min | 700 | 256 |
| 21% FB | | 1 | 1 | | 30 min | 0 | 273 |
| 21% FB | | 1 | 1 | | 30 min | 50 | 218 |
| 21% FB | | 1 | 1 | | 30 min | 0 | 275 |
| 21% FB | | 1 | 1 | | 30 min | 30 | 236 |
| 21% FB | 1 | 1 | | 0.018% SLS | 30 min | 0 | 233 |
| 21% FB | | 1 | 1 | 0.02% Benzalk Cl | 30 min | 0 | 237 |
| 21% FB | 1 | | | 0.1% SLS | 30 min | 0 | 163 |
| 21% FB | | 1 | 1 | 0.5% Lecithin | 30 min | 0 | 235 |
| 21% FB | 1 | | | 1% F68 | 30 min | 0 | 655 |
| 21% FB | 1 | 1 | | 1% PEG400 | 30 min | 0 | 308 |
| 21% FB | 1 | 1 | | 10% Trehalose | 30 min | 0 | 295 |
| 21% FB | 1 | 1 | | 10% Trehalose | 30 min | 0 | 236 |
| 21% FB | 1 | 1 | | 10% Trehalose | 30 min | 0 | 237 |
| 21% FB | 1 | 1 | | 5% Mannitol | 30 min | 0 | 247 |
| 21% FB | 1 | 0.5 | | 5% Mannitol | 30 min | 0 | 260 |
| 21% FB | 1 | 1 | | 5% Mannitol | 30 min | 0 | 247 |
| 21% FB | 1 | 1 | | 5% Mannitol | 30 min | 15 | 268 |
| 21% FB | 1 | 1 | | 5% Mannitol | 30 min | 44 | 278 |
| 21% FB | 1 | 1 | | 5% Mannitol | 30 min | 86 | 310 |
| 23% HCl | 1 | 1 | | | 3 hr | 0 | 122 |
| 23% HCl | 1 | 1 | | | 3 hr | 0 | 117 |
| 23% HCl | 1 | 1 | | | 3 hr | 4 | 120 |
| 23% HCl | 1 | 1 | | | 3 hr | 7 | 126 |
| 23% HCl | 1 | 1 | | | 3 hr | 52 | 142 |
| 23% HCl | 1 | 1 | | | 3 hr | 85 | 136 |
| 23% HCl | 1 | 1 | | | 3 hr | 123 | 142 |
| 23% HCl | 1 | | 1 | | 3 hr | 0 | 106 |
| 23% HCl | 1 | | 1 | | 3 hr | 17 | 113 |
| 23% HCl | 1 | | 1 | | 3 hr | 26 | 113 |
| 23% HCl | 1 | | 1 | | 3 hr | 48 | 122 |
| 23% HCl | 1 | | 1 | | 3 hr | 81 | 129 |
| 23% HCl | 1 | | 1 | | 3 hr | 119 | 120 |
| 23% HCl | 1 | | 1 | | 3 hr | 328 | 138 |
| 23% HCl | 1 | | 1 | | 3 hr | 700 | 160 |
| 23% HCl | 1 | 1 | | | 3 hr | 0 | 122 |
| 23% HCl | | 1 | 1 | | 3 hr | 0 | 122 |
| 23% HCl | | 1 | 1 | | 3 hr | 14 | 133 |
| 23% HCl | | 1 | 1 | | 3 hr | 45 | 161 |
| 23% HCl | | 1 | 1 | | 3 hr | 78 | 154 |
| 23% HCl | | 1 | 1 | | 3 hr | 116 | 144 |
| 23% HCl | | 1 | 1 | | 3 hr | 206 | 148 |
| 23% HCl | | 1 | 1 | | 3 hr | 325 | 157 |
| 23% HCl | | 1 | 1 | | 3 hr | 700 | 175 |
| 28% Mes | 2 | 0.5 | | | 6 hr | 0 | 376 |
| 28% Mes | 2 | 0.5 | | | 4 hr | 0 | 339 |
| 28% Mes | 2 | 0.5 | | | 3 hr | 0 | 406 |
| 28% Mes | 2 | 0.5 | | | 3 hr | 5 | 444 |
| 28% Mes | 2 | 0.5 | | | 3 hr | 50 | 415 |
| 28% Mes | 2 | 0.5 | | | 3 hr | 60 | 407 |
| 28% Mes | 2 | 0.5 | | | 3 hr | 92 | 518 |
| 28% Mes | 2 | 0.5 | | | 3 hr | 130 | 485 |
| 28% Mes | 2 | 0.5 | | | 3 hr | 339 | 525 |
| 28% Mes | 2 | 0.5 | | | 3 hr | 700 | 609 |
| 28% Mes | 2 | 0.5 | | | 6 hr | 0 | 376 |
| 28% Mes | 2 | 0.5 | | | 6 hr | 3 | 354 |
| 28% Mes | 2 | 0.5 | | | 120 min | 0 | 481 |
| 28% Mes | 2 | 0.5 | | | 120 min | 40 | 452 |
| 28% Mes | 2 | 0.5 | | | 120 min | 47 | 509 |

* Column 1 is ziprasidone compound - selected from free base, mesylate salt or hydrochloride salt

### Example 15

### Preparation of Formulation 1(42% Ziprasidone Free Base)

A coarse suspension was prepared by placing 21.92 gm of ziprasidone free base in the 100 ml milling chamber with 38.42 gm of milling media (500 micron polystyrene beads). To this, 10.44 ml of 10% Tween® 80 solution, 10.44 ml of 10% Pluronic® F108 solution and 5.22 ml of Lecithin were added. In addition, 13.8 ml of water for injection was added to the milling chamber. The above mixture was stirred until uniform suspension was obtained. This suspension was then milled for 80 minutes at 2100 RPM in a Nanomill-1 (Manufacturer Elan Drug Delivery, Inc.) and the temperature during milling was maintained at 4° C. The resulting suspension was filtered under vacuum to remove the milling media and the suspension characterized by microscopy and light scattering as described in example 1.

The filtered suspension was filled (2.5 ml) into flint vials. The vials were sealed with a rubber stopper and an aluminum seal was crimped on the stopper. The filled vials were sterilized for 15 min at 121° C. in a steam sterilizer. The suspension after sterilization was characterized and particle size measured by light scattering. The following table shows particle size stability of the 42% ziprasidone free base formulation after milling and following autoclaving.

TABLE D-7
Mean Particle Size of 42% Formulation I
After Milling and Following Autoclaving.

| | Mean particle size, |
|---|---|
| | D[4,3] (nm) |
| After milling | 262 nm |
| After Sterilization | 384 nm |

### Example 16

### Sterilization and Storage Stability of an Exemplary Formulation J Comprising 40% Ziprasidone Free Base

Formulation J was prepared as described in example 15. The filtered suspension was filled (3 ml) into flint vials. The vials were sealed with a rubber stopper and an aluminum seal was crimped on the stopper. The filled vials were sterilized for 15 min at 121° C. in a steam sterilizer. The suspension after sterilization was characterized and particle size measured by light diffraction. The filled vials were stored at 5, 25, and 40° C. and sampled at various times to determine particle size and stability of the suspension. The following table shows particle size stability of Formulation J during autoclaving and upon storage of the sterilized formulation.

TABLE D-8
Mean Particle Size of Formulation J after Autoclaving
and Upon Storage at 5, 25 and 40° C.

| | Mean particle size, |
|---|---|
| | D[4,3] (nm) |
| After milling | 291 nm |
| After Sterilization | 279 nm |

| Storage Time (days) | Temperature | Mean particle size, |
|---|---|---|
| post-sterilization | (° C.) | D[4,3] (nm) |
| 7 | 5 | 279 |
| 21 | 5 | 275 |
| 42 | 5 | 280 |
| 84 | 5 | 273 |
| 7 | 25 | 277 |
| 21 | 25 | 274 |
| 42 | 25 | 276 |
| 84 | 25 | 270 |
| 7 | 40 | 276 |
| 21 | 40 | 273 |
| 42 | 40 | 275 |
| 84 | 40 | 271 |

### Example 17

### Preparation of 21% Ziprasidone Free Base Formulation by High Pressure Homogenization and Storage Stability of the Formulation

A coarse suspension was prepared by placing pre-ground 17.71 gm ziprasidone freebase in 250 mL bottle with 8.4 mL of each, 10% w/v Pluronic F108 and 10% w/v Tween 80 and 55.6 mL of water. The suspension was placed in a cooling bath set to 5° C. The high pressure homogenizer (Manufacturer Avestin, Inc.) was cleaned and primed with water at full open setting. The suspension was pumped for three minutes under the full open condition of the homogenizer during which time it flowed smoothly. The pressure drop across the gap was then slowly increased until to 10,000 psi, and held for 5 minutes. The pressure drop across the gap was then increased to 15,000 psi, and was held here for 22 minutes. A sample of the homogenized suspension was taken at this point from the recirculation vessel, and homogenization was continued. The homogenization was stopped at 68 minutes at which time the formulation was pumped out of the homogenizer. The particle size of the final product samples was measured by laser diffraction (Malvern Mastersizer). The mean particle size (D[4,3]) of 21% ziprasidone free base formulation was 356 nm after homogenization. 2.7 ml of the above formulation and 0.3 mL of 5% w/v aqueous lecithin were filled into 5 mL vials and swirled to mix. All vials were stoppered and crimped and autoclaved for 15 minutes at 121° C. The autoclaved vials were placed in stability ovens and monitored for particle size. The particle size stability of the formulation is listed in the following table D-9.

TABLE D-9
Particle size stability of autoclaved 21% ziprasidone free base
nanosuspension prepared by high pressure homogenization.

| | | Mean Particle |
|---|---|---|
| Temperature | Time | Size (nm) |
| (degree C.) | (days) | D[4,3] |
| Before sterilization | 0 | 356 |
| After sterilization | 0 | 379 |
| 5 | 14 | 392 |
| 5 | 28 | 393 |
| 5 | 56 | 378 |
| 5 | 84 | 392 |
| | 0 | 379 |
| 30 | 14 | 383 |
| 30 | 28 | 384 |
| 30 | 56 | 380 |
| 30 | 84 | 379 |

### Example 18

### Preparation of a Dry Lyophilized 21% Ziprasidone Free Base Formulation

### Lyophilization Process

The 21% w/v Ziprasidone freebase nanosuspension was prepared by methods described in examples 7 and 8. Batch of 27% w/v Trehalose, 1% w/v F108, 1% w/v Tween 80, and 0.5% w/v Lecithin in water was used as diluent to prepare the samples for lyophilization. The formulation and diluent were combined in a ratio of 3 volumes of diluent to 1 volume of 21% formulation and were gently mixed. This resultant suspension was filled using a 0.5 mL fill volume into 5 mL and 10 mL glass vials and stoppered at the lyophilization position. These vials were then placed into the FTS LyoStar freeze-drying unit, and the following thermal program was run:
1) Shelves were cooled at 0.2° C./min (for 300 min) to −40° C. and held here for 120 min.
2) Shelves were warmed at 1° C./min (for 10 min) to −30° C. and 150 mTorr and held for 2000 min.
3) Shelves were warmed at 1° C./min (for 40 min) to 10° C. and 150 mTorr and held for 720 min.
4) Shelves were warmed at 1° C./min (for 20 min) to 30° C. and 150 mTorr and held for 720 min.
5) Shelves were cooled at 1° C./min (for 15 min) to 15° C. and 150 mTorr and held until cycle could be manually ended.

The freeze-drying cycle was manually stopped, and the vials were stoppered and crimped. They were then placed in the refrigerator for storage.

The dry cake in the vials were reconstituted with the same volume as the initial fill with either 0.5 mL of water or 0.5 mL of 1% w/v F108, 1% w/v Tween80, 0.5% w/v Lecithin in water (the formulation vehicle). These vials were swirled, upon which the cake wetted and reconstituted into a milky white suspension easily.

In order to determine if this lyophile could also be reconstituted to a higher concentration, the cake was reconstituted with 0.125 mL of water to result in 21% concentration equivalent to the initial drug level. The cake wetted and reconstituted into suspension easily as well. The reconstituted suspensions were then analyzed for particle size by Laser Diffraction. The particle size results are listed in the following Table D-10. A refrigerated, non-lyophilized suspension served as the control.

TABLE D-10
Particle sizing of reconstituted Ziprasidone freebase lyophiles

| | Volume of vehicle | Sonication for | Mean Particle |
|---|---|---|---|
| Vehicle for | used for | p. size | Size (nm) |
| Reconstitution | reconstitution | measurement? | D[4,3] |
| Control-none | N/A | No | 292 |
| Water | 0.5 mL | No | 467 |
| Water | 0.5 mL | Yes | 382 |
| Stabilizer | 0.5 mL | No | 464 |
| solution | | | |
| Stabilizer | 0.5 mL | Yes | 385 |
| solution | | | |
| Water | 0.125 mL | No | 471 |
| Water | 0.125 mL | Yes | 358 |

### Example 19

### Pharmacokinetic Study in Dogs Comparing Unmilled and Micronized Ziprasidone Free Base and its Salts to Ziprasidone Free Base and Salt Nanoparticles

Pharmacokinetic studies were conducted with various particle sizes of ziprasidone freebase, and its salts in aqueous suspension formulations to determine the effect of particle size on PK performance of the drug in-vivo. Ziprasidone free base and salt formulations with a mean effective diameter of less than 1000 nm showed significantly higher exposure (Average depot levels and Area under the curve) than a formulations with particle size greater than 5 μm (higher AUC and average depot levels). See Table D-11, presented in Working Examples 1-16.

TABLE D-11
Pharmacokinetics of Ziprasidone in Dog Following IM Administration of Various
Depot Formulations. Reported values are mean ± sd of n = 4 dogs.

| | Effective | | | Average | |
|---|---|---|---|---|---|
| | diameter or | Dose of | | Depot | |
| | mean | Ziprasidone | | (C_{1-3 wk}) | |
| | diameter | active | AUC_{0-inf} | Levels | Cₘₐₓ |
| Formulation | (nm) | (mg) | (ng · h/ml) | (ng/ml) | (ng/ml) |
| 42% Ziprasidone | 384 | 840 | 117408 ± 31097 | 243 ± 86 | 495 ± 98 |
| Free Base with 2% | | | | | |
| Pluronic F108, 2% | | | | | |
| Tween 80 and 0.5% | | | | | |
| Lecithin | | | | | |
| 21% Ziprasidone | 260 | 420 | 58300 ± 6490 | 110 ± 23 | 146 ± 35 |
| Free Base with 2% | | | | | |
| PVP and 0.1% SLS | | | | | |
| 21% Ziprasidone | 231 | 420 | 62600 ± 9400 | 100 ± 15 | 180 ± 85 |
| Free Base with 1% | | | | | |
| Pluronic F108 and | | | | | |
| 1% Tween 80 | | | | | |
| 21% Ziprasidone | 911 | 420 | 64400 ± 7800 | 105 ± 19 | 389 ± 231 |
| Free Base with 1% | | | | | |
| Pluronic F108, 1% | | | | | |
| Tween 80 and 0.5% | | | | | |
| Lecithin | | | | | |
| 23% Ziprasidone | 113 | 420 | 53800 ± 11000 | 78 ± 14 | 211 ± 168 |
| Hydrochloride salt | | | | | |
| with 1% Pluronic | | | | | |
| F108 and 1% PVP | | | | | |
| 28% Ziprasidone | 406 | 420 | 48700 ± 4400 | 74 ± 14 | 116 ± 39 |
| Mesylate Salt | | | | | |
| 2% PVP and | | | | | |
| 0.5% Pluronic F108 | | | | | |
| 21% Micronized | 4660 | 420 | 40000 ± 6700 | 47 ± 8 | 71 ± 14 |
| Ziprasidone Free | | | | | |
| Base, 1.5% | | | | | |
| NaCMC and 0.1% | | | | | |
| Tween 80 aqueous | | | | | |
| suspension | | | | | |
| 28% Micronized | 3610 | 420 | 38900 ± 1600 | 55 ± 27 | 73 ± 40 |
| Ziprasidone | | | | | |
| Mesylate salt, | | | | | |
| 0.1% Tween 80 | | | | | |
| aqueous suspension | | | | | |
| 28% Ziprasidone | 10660 | 420 | 31400 ± 1100 | 43 ± 30 | 60 ± 38 |
| Mesylate-Nominal | | | | | |
| size aqueous | | | | | |
| suspension | | | | | |

All mentioned documents are incorporated by reference as if here written. When introducing elements of the present invention or the exemplary embodiment(s) thereof, the articles “a,” “an,” “the” and “said” are intended to mean that there are one or more of the elements. The terms “comprising,” “including” and “having” are intended to be inclusive and mean that there may be additional elements other than the listed elements. Although this invention has been described with respect to specific embodiments, the details of these embodiments are not to be construed as limitations.

1. An injectable depot pharmaceutical formulation comprising:
a) a pharmaceutically effective amount of a compound, the compound in the form of nanoparticles having an average particle size of less than about 2000 nm;
b) a pharmaceutically acceptable carrier; and
c) at least two surface stabilizers;
wherein the compound has low water solubility; and wherein at least one of the surface stabilizers is adsorbed on the surface of the nanoparticles, and wherein the combined amount of the surface stabilizers is effective to maintain the average particle size of the nanoparticles.

2. The formulation according to claim 1, wherein at least two of the surface stabilizers are adsorbed on the surface of the nanoparticles.

3. The formulation as in claim 1, wherein the compound has a logP of at least about 3 or greater.

4. The formulation as in claim 1, wherein the compound is crystalline.

5. The formulation as in claim 1, wherein the carrier is water.

6. The formulation as in claim 1, wherein the nanoparticles have an average particle size of less than about 1500 nm.

7. The formulation as in claim 1, wherein the amount by weight of one of the two surface stabilizers is from about 0.5% to about 3.0% by weight of the total volume of the formulation, and the amount by weight of the other surface stabilizer is from about 0.1% to about 3.0% by weight of the total volume of the formulation.

8. The formulation according to claim 7, comprising a third surface stabilizer, wherein the amount by weight of the third surface stabilizer is from about 0.018% to about 1.0% by weight of the total volume of the formulation.

9. The formulation as in claim 1, wherein one of the two surface stabilizers is selected from the group consisting of crystallization inhibitors, anionic surfactants, cationic surfactants, amphoteric surfactants, non-ionic surfactants and polymeric surfactants; and the other surface stabilizer is selected from the group consisting of anionic surfactants, cationic surfactants, amphoteric surfactants, non-ionic surfactants and polymeric surfactants.

10. The formulation of claim 1, further comprising a bulking agent.

11. An injectable depot pharmaceutical formulation comprising:
a) a pharmaceutically effective amount of a compound of low water solubility, the compound in the form of nanoparticles having an average particle size of less than about 1200 nm;
b) water;
c) a first surface stabilizer adsorbed on the surface of the nanoparticles; and
d) a second surface stabilizer;
wherein the amount by weight of the compound is from about 20% by weight to about 60% by weight of the total volume of the formulation;
wherein the amount by weight of a first surface stabilizer is from about 0.5% to about 2.0% by weight of the total volume of the formulation;
wherein the amount by weight of a second surface stabilizer is from about 0.1% to about 2.0% by weight of the total volume of the formulation; and wherein amount of the first surface stabilizer and the amount of the second surface stabilizer are together effective to maintain the average particle size of the nanoparticles.

12. The formulation according to claim 11, wherein the second surface stabilizer is adsorbed on the surface of the nanoparticles.

13. The formulation as in claim 11, wherein the compound has a logP of at least about 3 or greater.

14. An injectable depot pharmaceutical formulation comprising:
a) a pharmaceutically effective amount of a compound, the compound in the form of nanoparticles having an average particle size of less than about 2000 nm;
b) a pharmaceutically acceptable carrier; and
c) at least two surface stabilizers;
wherein the compound has a logP of at least about 3 or greater; and wherein at least one of the surface stabilizers is adsorbed on the surface of the nanoparticles, and wherein the combined amount of the surface stabilizers is effective to maintain the average particle size of the nanoparticles.

15. An injectable depot pharmaceutical formulation comprising:
a) a pharmaceutically effective amount of a compound, the compound in the form of nanoparticles having an average particle size of less than about 2000 nm;
b) a pharmaceutically acceptable carrier; and
c) at least two surface stabilizers;
wherein the compound has a high melting point; and wherein at least one of the surface stabilizers is adsorbed on the surface of the nanoparticles, and wherein the combined amount of the surface stabilizers is effective to maintain the average particle size of the nanoparticles.
